(19) United States
(12) Patent Application Publication
(10) Pub. No.: US 2026/0048020 A1
(43) Pub. Date: Feb. 19, 2026

(54) GELATIN BASED URETHANE/UREA MICROCAPSULES

(71) Applicant: ENCAPSYS, LLC, Appleton, WI (US)

(72) Inventors: Todd Arlin Schwantes, Lena, WI (US); Nianxi Yan, Appleton, WI (US)

(73) Assignee: ENCAPSYS, LLC, Appleton, WI (US)

(21) Appl. No.: 19/367,772

(22) Filed: Oct. 23, 2025

Related U.S. Application Data
(63) Continuation of application No. 17/962,481, filed on Oct. 8, 2022.
(60) Provisional application No. 63/254,384, filed on Oct. 11, 2021.

(51) Int. Cl. A61K 9/50; C08G 18/72; C11D 17/00

(52) U.S. Cl. CPC A61K 9/5057; C08G 18/72; C11D 17/0039

(57) ABSTRACT

Core shell microcapsules are provided wherein the capsule shell is an interfacial copolymer formed of a gelatin and an isocyanate.

### RELATED APPLICATIONS

The present application is a continuation of pending U.S. patent application Ser. No. 17/962,481, filed Oct. 8, 2022 which claims the benefit of prior U.S. Provisional Patent Application No. 63/254,384, filed Oct. 11, 2021, both entitled “Gelatin Based Urethane/Urea Microcapsules”, the contents of both of which are hereby incorporated herein by reference in their entirety.

### FIELD

The present teaching relates to capsule manufacturing processes and microcapsules produced by such processes, along with improved articles of manufacture based on such microcapsules.

### DESCRIPTION OF THE RELATED ART

Various processes for microencapsulation, and exemplary methods and materials are set forth in a multitude of patents, such as Schwantes (U.S. Pat. No. 6,592,990), Nagai et al. (U.S. Pat. No. 4,708,924), Baker et al. (U.S. Pat. No. 4,166,152), Wojciak (U.S. Pat. No. 4,093,556), Matsukawa et al. (U.S. Pat. No. 3,965,033), Matsukawa (U.S. Pat. No. 3,660,304), Ozono (U.S. Pat. No. 4,588,639), Irgarashi et al. (U.S. Pat. No. 4,610,927), Brown et al. (U.S. Pat. No. 4,552,811), Scher (U.S. Pat. No. 4,285,720), Hayford (U.S. Pat. No. 4,444,699), Shioi et al. (U.S. Pat. No. 4,601,863), Kiritani et al. (U.S. Pat. No. 3,886,085), Jahns et al. (U.S. Pat. Nos. 5,596,051 and 5,292,835), Matson (U.S. Pat. No. 3,516,941), Chao (U.S. Pat. No. 6,375,872), Foris et al. (U.S. Pat. Nos. 4,001,140; 4,087,376; 4,089,802 and 4,100,103) and Greene et al. (U.S. Pat. Nos. 2,800,458; 2,800,457 and 2,730,456), among others and as taught by Herbig in the chapter entitled “Microencapsulation” in Kirk-Othmer Encyclopedia of Chemical Technology, V.16, pages 438-463.

Other useful methods for microcapsule manufacture are: Foris et al. (U.S. Pat. Nos. 4,001,140 and 4,089,802) describing a reaction between urea and formaldehyde; Foris et al. (U.S. Pat. No. 4,100,103) describing reaction between melamine and formaldehyde; and Fuji Photo Film Co, (GB No. 2,062,570) describing a process for producing microcapsules having walls produced by the polymerization of melamine and formaldehyde in the presence of a styrene sulfonic acid. Alkyl acrylate-acrylic acid copolymer capsules are taught in Brown et al. (U.S. Pat. No. 4,552,811). Each patent described throughout this application is incorporated herein by reference to the extent each provides guidance regarding microencapsulation processes and materials.

Interfacial polymerization is a process wherein a microcapsule wall of polyamide, an epoxy resin, a polyurethane, a polyurea or the like is formed at an interface between two phases. Riecke (U.S. Pat. No. 4,622,267) discloses an interfacial polymerization technique for preparation of microcapsules in which the core material is initially dissolved in a solvent and an aliphatic diisocyanate soluble in the solvent mixture is added. Subsequently, a nonsolvent for the aliphatic diisocyanate is added until the turbidity point is just barely reached. This organic phase is then emulsified in an aqueous solution, and a reactive amine is added to the emulsion. The amine diffuses to the interface, where it reacts with the diisocyanate to form polymeric polyurethane shells. A similar technique, used to encapsulate salts which are sparingly soluble in water in polyurethane shells, is disclosed in Greiner et al. (U.S. Pat. No. 4,547,429). Matson (U.S. Pat. No. 3,516,941) teaches polymerization reactions in which the material to be encapsulated, the “core material,” is dissolved in an organic, hydrophobic oil phase which is dispersed under high shear mixing in an aqueous phase to form a dispersion of fine oil droplets. The aqueous phase has dissolved therein aminoplast precursor materials, namely, an amine and an aldehyde, which upon polymerization form the wall of the microcapsule. Polymerization is initiated by the addition and initiation of an acid catalyst which results in the formation of an aminoplast polymer which is insoluble in both phases. As the polymerization advances, the aminoplast polymer separates from the aqueous phase and deposits on the surface of the dispersed droplets of the oil phase where polymerization continues to form a capsule wall at the interface of the two phases, thus encapsulating the core material. Urea-formaldehyde (UF), urea-resorcinol-formaldehyde (URF), urea-melamine-formaldehyde (UMF), and melamine-formaldehyde (MF), capsule formations proceed in a like manner. Depending upon the selection of wall forming materials and the encapsulation steps chose, oftentimes each phase, the oil phase and the water phase, contains at least one of the capsule wall-forming materials wall and polymerization occurs at the phase boundary. Thus, a polymeric capsule shell wall forms at the interface of the two phases thereby encapsulating the core material. Wall formation of polyester, polyamide, and polyurea capsules also typically proceed via interfacial polymerization.

Common microencapsulation processes can be viewed as a series of steps. First, the core material which is to be encapsulated is typically emulsified or dispersed in a suitable dispersion medium. This medium is typically aqueous but involves the formation of a polymer rich phase. Most frequently, this medium is a solution of the intended capsule wall forming material, or at least one component thereof. The solvent characteristics of the medium are changed such as to cause phase separation of the wall forming material whereby the wall forming material is contained in a discrete liquid phase which is also dispersed in the same medium as the intended capsule core material. The dispersed droplets of the wall forming material deposit themselves as a continuous coating on the surface of the dispersed droplets of the internal phase or core material. The wall forming material is then solidified. This process is commonly known as coacervation.

Turning to the present teaching, the use of gelatin in encapsulation processes, whether in sheet or like form or as discrete capsules or microcapsules is well known and is perhaps one of, if not the first, encapsulating agents. For example, with respect to the former, Yutzy et. al. (U.S. Pat. No. 2,614,928) and Lowe et. al. (U.S. Pat. No. 2,613,930) describe the use of gelatins and modified gelatins, especially the latter which are reacted with sulfonyl chlorides, carboxylic acid chlorides, acid anhydrides, mono-isocyanates and the like, as materials for enveloping or encasing silver halide in a mass of the gelatin in preparing photographic emulsions. With respect to the latter, Matsukawa et. al. (U.S. Pat. No. 3,994,502) described the production of microcapsules by the reaction of gum arabic and gelatin wherein the gelatins were modified gelatins, including those taught in the aforementioned Yutzy et. al. and Lowe et. al. Vassiliades et. al. (U.S. Pat. No. 4,138,362) describe various microcapsules for use in the encapsulation of oily substances for pressure responsive, image transfer applications wherein the microcapsules are formed by the reaction of natural amine and imine containing polymers including gelatin, chitin, chitosan and the like with polyisocyanates. In the one example using polyisocyanate and gelatin both wall forming components were present in the same amount, a 1:1 weight ratio. Finally, and Kamiya (U.S. Pat. No. 8,871,347) describes microencapsulated latent type curing agents wherein the shell wall is formed of polyisocyanates and enzyme treated gelatins wherein the gelatin is employed in an amount of 1 to 100, preferably 10 to 50, parts by mass per 100 parts by mass of the polyisocyanates. It is also to be appreciated that gelatin has been employed in a number of microencapsulation processes as a modifier or added emulsifier at low levels as compared to the wall forming material, particularly the isocyanate components.

Despite the advances and achievements with gelatin encapsulation, even with gelatin/isocyanate microcapsules, there is still a need and desire for improved properties combined with more environmentally suitable microcapsules. In particular, there is a continuing need and desire to develop microcapsules with the beneficial strength and controlled release capabilities associated with isocyanate-based microcapsules, if not with improved capabilities over existing isocyanate-based microcapsules, while providing a more safe and environmentally friendly process by reducing the amount of isocyanates and more environmentally responsible microcapsule having improved natural degradation.

### SUMMARY

The present teaching relates to microcapsules formed by any suitable oil-in-water microencapsulation process, especially interfacial polymerization, comprising a core material and a shell encapsulating the core material wherein the shell comprises the reaction product of a) gelatin derived from an aqueous phase and b) an isocyanate component comprising two or more aliphatic di- and/or poly-isocyanates, two or more aromatic di- and/or poly-isocyanates, or a mixture of at least one aliphatic di- and/or poly-isocyanate and at least one aromatic di- and/or poly-isocyanate, derived from an oil phase wherein each of the minimum required isocyanates being present at a level of at least 10 mole percent based on the total isocyanate content and the weight ratio of gelatin to isocyanate is from 1:0.5 to 1:0.01, preferably from 1:0.35 to 1:0.05, most preferably from 1:0.20 to 1:0.1. Preferably, the isocyanates are or are predominantly di-isocyanates and/or tri-isocyanates and/or at least one of the isocyanates is a biuret and/or adduct of a di-isocyanate and/or tri-isocyanate, most especially adducts thereof with one or more triols, most especially trimethylolpropane. Most preferably, the isocyanate component is a mixture of aliphatic and aromatic isocyanates.

The core material typically and preferably comprises a benefit agent. Exemplary benefit agents include perfumes, fragrances, agricultural actives, phase change materials, essential oils, lubricants, colorants, preservatives, antimicrobial actives, antifungal actives, herbicides, antiviral actives, antiseptic actives, antioxidants, biological actives, deodorants, antiperspirant actives, emollients, humectants, exfoliants, ultraviolet absorbing agents, corrosion inhibitors, silicone oils, waxes, bleach particles, fabric conditioners, malodor reducing agents, dyes, optical brighteners and mixtures thereof.

According to second aspect of the present teaching there is provided an oil-in water microencapsulation process for the formation of the aforementioned microcapsules. Generally speaking, there is provided an oil-in-water microencapsulation process wherein an oil phase comprising a core material and an isocyanate wall forming component comprising two or more aliphatic di- and/or poly-isocyanates, two or more aromatic di- and/or poly-isocyanate, or a mixture of one or more aliphatic di- and/or poly-isocyanates and one or more aromatic di- and/or poly-isocyanates, each of the minimum required isocyanates being present at a level of at least 10 mole percent based on the total isocyanate content, is dispersed in an aqueous phase comprising a gelatin co-reactive therewith wherein the weight ratio of gelatin to isocyanate is from 1:0.5 to 1:0.01, preferably from 1:0.35 to 1:0.05, most preferably from 1:0.20 to 1:0.1.

Finally, according to a third embodiment there are provided articles of manufacture incorporating the aforementioned microcapsules. Exemplary articles of manufacture include, but are not limited to soaps, surface cleaners, laundry detergents, fabric softeners, shampoos, textiles, paper products including tissues, towels, napkins, and the like, adhesives, wipes, diapers, feminine hygiene products, facial tissues, pharmaceuticals, deodorants, heat sinks, foams, pillows, mattresses, bedding, cushions, cosmetics and personal care products, medical devices, packaging, agricultural products, coolants, wallboard, insulation, and the like.

The microcapsules formed according to the present teaching provide i) excellent properties, both in terms of physical properties and performance, such as shell strength, integrity and leakage, ii) the ability to protect, retain or deliver a benefit agent to a targeted situs and/or on a controlled basis, and/or ill) superior degradability despite the marked low level of isocyanate. Surprisingly, it has now been found that gelatin/isocyanate microcapsules may be formed using markedly lower levels of isocyanate than previously believed and known without, from a commercial perspective, compromising or significantly compromising the properties of traditional gelatin/isocyanate microcapsules and, depending upon the embodiment with improved properties. Beyond their physical attributes and properties, these microcapsules have certain beneficial attributes as well. Specifically, these microcapsules and their method of production have a marked benefit from an economic, environmental and health and safety perspective due to the marked reduction in the need for and levels of use of isocyanates which are associated with a plethora of environmental, health and safety concerns. Furthermore, the resulting microcapsules also have excellent and improved degradability and tend to be less costly due to the shift to the more cost-effective gelatin wall forming materials.

### DETAILED DESCRIPTION

According to the present there are provided microcapsules formed by any suitable oil-in-water microencapsulation process, especially interfacial polymerization, comprising a core material and a shell encapsulating the core material wherein the shell comprises the reaction product of a) gelatin derived from an aqueous phase and b) an isocyanate component comprising two or more aliphatic di- and/or poly-isocyanates, two or more aromatic di- and/or poly-isocyanates, or a mixture of at least one aliphatic di- and/or poly-isocyanate and at least one aromatic di- and/or poly-isocyanate, derived from an oil phase, each of the minimum required isocyanates being present at a level of at least 10 mole percent based on the total isocyanate content, wherein the weight ratio of gelatin to isocyanate is from 1:0.5 to 1:0.01, preferably from 1:0.35 to 1:0.05, most preferably from 1:0.20 to 1:0.1.

Gelatins suitable for use in the practice of the present teaching are well known and widely available and are broadly used across a number of industries for encapsulation of, e.g., food substances, pharmaceuticals, cosmetics, agricultural products and the like. Gelatin is typically prepared either by partial acid (gelatin type A) or alkaline hydrolysis (gelatin type B) of native collagen that is found in animal collagen from skins, cartilage, bones, and tendons, especially those of fish, cattle, swine, chickens, and the like. The surface of gelatin is negatively charged at higher pH and positively charged at lower pH (pH 5). The isoelectric point of gelatin A is in the region of 8-9, while it is about pH 4.8 to 5.4 for gelatin type B. Gelatin for use in the present teaching also includes gelatin hydrolysates prepared by hydrolysis of gelatin with a protease such as collagenase and cysteine protease. Suitable gelatins will typically have a Bloom value of from 55 to 325.

The weight average molecular weight of the gelatin used in the present invention is not particularly limited as long as the effects of the invention are not impaired for the given end-use application. Generally speaking, the weight average molecular weight is from about 5,000 to about 80,000, preferably from about 10,000 to about 65,000, more preferably from about 15,000 to about 40,000. Higher, up to 110,000 weight average molecular weights, and lower, as low as 1,000 weight average molecular weight, gelatins may be used; however, one has to be concerned with functionality depending upon the end use since the higher the weight average molecular weight the more sticky the gelatin and, hence, the microcapsules, particularly in the presence of moisture and the lower the weight average molecular weight the more difficult it is to retain the shape and physical properties of the microcapsules. Of course, one can adjust properties by employing two or more kinds of gelatins having different weight average molecular weights; however, in such instances it is preferred that the overall weight average molecular weight of the combination is adjusted to fall within the numerical range, particularly the preferred ranges, described above. Typically, the weight average molecular weight of the gelatin is determined in accordance with the methods specified in “20-1 Molecular weight distribution” and “20-2 Average molecular weight” in “PAGI METHOD, Tenth edition” (Commission on Testing Method for Photographic Gelatin, 2006).

In preparing the gelatin-containing aqueous phase it is often preferable to use a gelatin which has undergone an acid treatment as such gelatins often facilitate or enable better control on particle size, particularly if one is seeking to control particle size to a single-digit micrometer order. Similarly, from the perspective of gel network formation, it is preferred to use a gelatin having a relatively low jelly strength. Specifically, it is preferred to use a gelatin which exhibits a jelly strength according to JIS K6503-2001 of 10 to 200.

Though not a necessity, distilled water and deionized water are preferably used as the water for the aqueous phase. The concentration of the gelatin in the aqueous phase is typically from 0.1 to 40%, preferably from 0.1 to 15%, more preferably from 1 to 10% by weight based on the combined weight of the gelatin and water. If the content of gelatin with respect to water is too low, emulsification is destabilized, while if the content is too high, the emulsion dispersibility deteriorates.

### Isocyanate

The second critical wall forming component of the microcapsules of the present teaching is the isocyanate component. As used herein the term “isocyanate” is used interchangeably with the term “polyisocyanate” and refers to such materials having two or more isocyanate groups, i.e., —N═C═O. Although mono-isocyanates may be used in combination with the herein recited required isocyanates, the critical and required isocyanates have at least two isocyanate groups. As noted, the isocyanate component comprises two or more aliphatic di- and/or poly-isocyanates, two or more aromatic di- and/or poly-isocyanates, or a mixture of at least one aliphatic di- and/or poly-isocyanate and at least one aromatic di- and/or poly-isocyanate, wherein each of the minimum required isocyanates is present at a level of at least 10 mole percent based on the total isocyanate content. Specifically, with respect to wholly aliphatic or wholly aromatic isocyanates, if three or more aliphatic or aromatic isocyanates are used, only two must be present in at least 10 mole percent. With respect to the mixture of aliphatic and aromatic isocyanates, at least one of each much be present at 10 mole percent. Preferably the mole ratio between the two required isocyanates is from 90:10 to 10:90, preferably 75:25 to 25:72, more preferably 65:35 to 35:65 in the case of wholly aromatic or wholly aliphatic isocyanates and, in the case of mixed aliphatic and aromatic isocyanates, the mole ratio of aliphatic to aromatic isocyanate is typically from 90:10 to 10:90, preferably from 80:20 to 20:80, more preferably from 70:30 to 30:70, most preferably from 65:35 to 50:50.

Suitable isocyanates can be aromatic or aliphatic; linear, branched, or cyclic and include the monomeric, dimer, trimer, biuret forms as well as oligomers and prepolymers thereof. Particularly desirable are oligomers and prepolymers thereof (i.e., adducts) with other compounds reactive with the isocyanate groups (i.e., —N═C═O), e.g., diols, triols, diamines, triamines and the like, in which at least one, preferably at least two, of the reactive groups of said other compounds are reacted with and thereby carry an isocyanate monomer, dimer, trimer or biuret, especially isocyanate adducts formed with one or more polyols, particularly one or more diols or triols, most especially trimethylol propane (TMP). Though not limited thereto, the isocyanates typically have, on average, 2 to 4 isocyanate groups. Preferably, the isocyanates are wholly or predominantly di-isocyanates and/or tri-isocyanates and/or at least one of the isocyanates is a biuret and/or adduct thereof, particularly adducts thereof with one or more diols, triols, diamines, or triamines, especially diols and triols, most especially trimethylolpropane adducts thereof. In this respect, at least 50 mole percent, preferably at least 65 mole percent, most preferably at least 75 mole percent of the isocyanates are di-isocyanates and/or adducts thereof. Most preferably, the isocyanate component is a mixture of aliphatic and aromatic isocyanates.

Suitable aliphatic isocyanates include hexamethylene diisocyanate, dicyclohexyl-methyl diisocyanate, isophorone diisocyanate, hydrogenated xylylene diisocyanate, as well as their respective trimers and biurets such as the trimer of hexamethylene diisocyanate, the trimer of isophorone diisocyanate and the biuret of hexamethylene diisocyanate. Exemplary commercially available aliphatic isocyanates include, e.g., DESMODUR W which is dicyclohexylmethane diisocyanate; DESMODUR N3600, DESMODUR N3700, and DESMODUR N3900, which are low viscosity, polyfunctional aliphatic polyisocyanates based on hexamethylene diisocyanate; and DESMODUR 3600 and DESMODUR N100 which are aliphatic polyisocyanates based on hexamethylene diisocyanate, each of which is available from Covestro AG.

Suitable aromatic isocyanates include those having phenyl, tolyl, xylyl, naphthyl or diphenyl moiety as the aromatic component. Exemplary aromatic isocyanates include a polyisocyanurate of toluene diisocyanate, a trimethylol propane-adduct of toluene diisocyanate or a trimethylol propane-adduct of xylylene diisocyanate. One class of suitable aromatic isocyanates are the polyisocyanates having the generic structure:

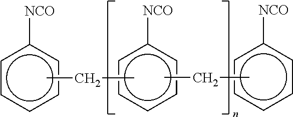

(i.e., polymeric methylene diphenyl diisocyanate or PMDI, and its structural isomers) wherein n can vary from zero to a desired number, preferably n is less than 6. Mixtures of these polyisocyanate are also suitable wherein the value of n can vary from 0 to 6 with an average value of n falls in between 0.5 and 1.5.

Specific examples of wall forming monomer isocyanates include, for example, 1,5-naphthylene diisocyanate, 4,4′-diphenylmethane diisocyanate (MDI), hydrogenated MDI (H12MDI), xylylene diisocyanate (XDI), tetramethylxylol diisocyanate (TMXDI), 4,4′˜ diphenyldimethylmethane diisocyanate, di- and tetraalkyldiphenyl-methane diisocyanate, 4,4′-dibenzyl diisocyanate, 1,3-phenylene diisocyanate, 1,4-phenylene diisocyanate, the isomers of toluene diisocyanate (TDI), optionally in a mixture, 1-methyl-2,4-diisocyanatocyclohexane, 1,6-diisocyanato-2,2,4-trimethyl-hexane, 1,6-diisocyanato-2,4,4-trimethylhexane, 1-isocyanatomethyl-3-isocyanato-1,5,5-trimethylcyclohexane, 4,4′-diisocyanatophenylperfluoroethane, tetramethoxybutane 1,4-diisocyanate, butane 1,4-diisocyanate, chlorinated and brominated diisocyanates, phosphorus-containing diisocyanates, hexane 1,6-diisocyanate (HDI), dicyclohexylmethane diisocyanate, cyclo-hexane 1,4-diisocyanate, ethylene diisocyanate, phthalic acid bisisocyanatoethyl ester, also polyisocyanates with reactive halogen atoms, such as 1-chloromethylphenyl 2,4-diisocyanate, 1-bromomethylphenyl 2,6-diisocyanate, 3,3-bischloromethyl ether 4,4′-diphenyldiisocyanate, trimethylhexamethylene diisocyanate, 1,4-diisocyanatobutane, 1,2-diisocyanatododecane and dimer fatty acid diisocyanate.

Other suitable commercially-available polyisocyanates include LUPRANATE M20 (polymeric methylene diphenyl diisocyanate, “PMDI” commercially available from BASF containing isocyanate group “NCO” 31.5 wt %), where the average n is 0.7; PAPI 27 (PMDI commercially available from Dow Chemical having an average molecular weight of 340 and containing NCO 31.4 wt %) where the average n is 0.7; MONDUR MR (PMDI containing NCO at 31 wt % or greater, commercially available from Covestro AG) where the average n is 0.8; MONDUR MR Light (PMDI containing NCO 31.8 wt %, commercially available from Covestro AG) where the average n is 0.8; MONDUR 489 (PMDI commercially available from Covestro AG containing NCO 30-31.4 wt %) where the average n is 1.0; poly[(phenylisocyanate)-co-formaldehyde] (Aldrich Chemical, Milwaukee, Wis.), other isocyanate monomers such as DESMODUR N3200 (poly(hexamethylene diisocyanate) commercially available from Covestro AG), and TAKENATE D110-N (xylene diisocyanate adduct polymer commercially available from Mitsui Chemicals corporation, Rye Brook, N.Y., containing NCO 11.5 wt %).

In particular embodiments, an exemplary isocyanate has the following structure:

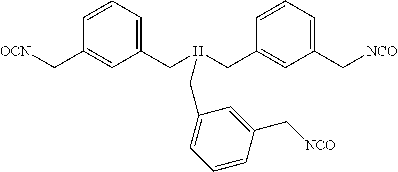

or its structural isomer. Representative isocyanates are TAKENATE D-110N (an isocyanate adduct based on xylene diisocyanate commercially available from Mitsui), DESMODUR L75 (an isocyanate adduct based on toluene diisocyanate commercially available from Covestro AG), and DESMODUR IL (another trimer isocyanate based on toluene diisocyanate available from commercially Covestro AG).

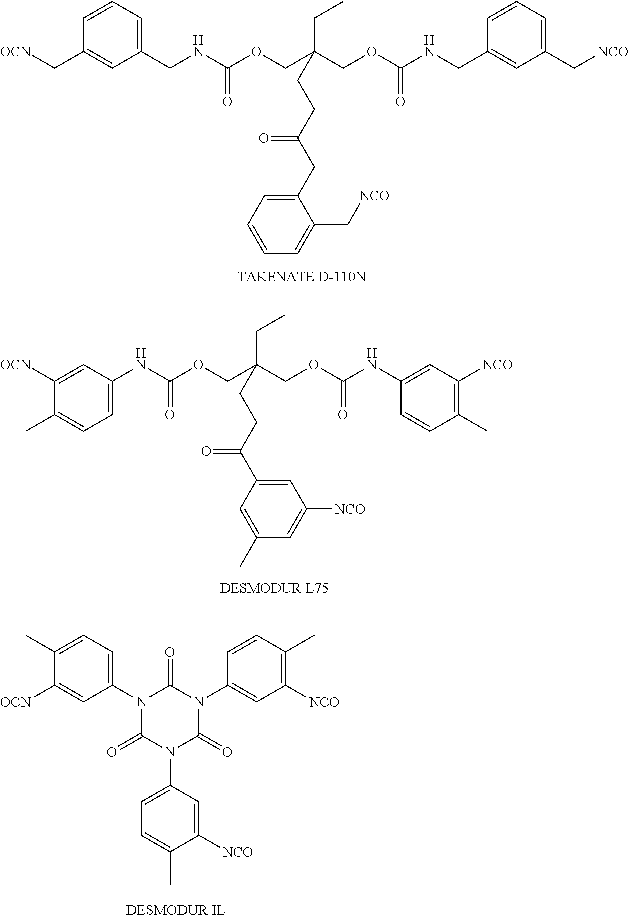

More examples of suitable isocyanates can be found in PCT 2004/054362; EP 0 148149; EP 0 017 409 B1; U.S. Pat. Nos. 4,417,916, 4, 124,526, 5,583,090, 6,566,306, 6,730,635, PCT 90/08468, PCT WO 92/13450, U.S. Pat. Nos. 4,681,806, 4,285,720 and 6,340,653.

The average molecular weight of certain isocyanates useful in this invention varies from 250 to 1000 Da and preferably from 275 to 500 Da: though higher molecular weights are useful and expected in the case of isocyanate oligomers/adducts and prepolymers. In general, the range of the isocyanate concentration in the oil phase varies from 0.1% to 10%, preferably from 0.1% to 8%, more preferably from 0.2 to 5%, and even more preferably from 1.5% to 3.5%, all based on the combined weight of the isocyanate and core composition, i.e., diluent and core material.

### Core/Benefit Material

The capsules according to the present teaching are useful with a wide variety of capsule contents (“core materials” or “benefit agents”) including, by way of illustration and without limitation, internal phase oils, solvent oils, phase change materials, lubricants, dyes, cleaning oils, polishing oils, flavorings, nutrients, sweeteners, chromogens, pharmaceuticals, fertilizers, herbicides, biological actives, scents, perfumes, fragrances, agricultural actives, essential oils, colorants, preservatives, antimicrobial actives, antifungal actives, herbicides, antiviral actives, antiseptic actives, antioxidants, biological actives, deodorants, antiperspirant actives, emollients, humectants, exfoliants, ultraviolet absorbing agents, corrosion inhibitors, silicone oils, waxes, bleach particles, fabric conditioners, malodor reducing agents, optical brighteners, perfume raw materials, such as alcohols, ketones, aldehydes, esters, ethers, nitriles, alkenes, fragrance solubilizers, preservatives, self-healing compositions, higher fatty acids, lipids, skin coolants, vitamins, sunscreens, glycerin, catalysts, silicon dioxide particles, brighteners, antibacterial actives, cationic polymers and mixtures of any two or more of the foregoing. Exemplary phase change materials useful as core materials include, by way of illustration and not limitation, paraffinic hydrocarbons having 13 to 28 carbon atoms, various hydrocarbons such n-octacosane, n-heptacosane, n-hexacosane, n-pentacosane, n-tetracosane, n-tricosane, n-docosane, n-heneicosane, n-eicosane, n-nonadecane, octadecane, n-heptadecane, n-hexadecane, n-pentadecane, n-tetradecane, n-tridecane. Additional or alternative phase change materials include crystalline materials such as 2,2-dimethyl-1,3-propanediol, 2-hydroxymethyl-2-methyl-1,3-propanediol, acids of straight or branched chain hydrocarbons such as eicosanoic acid and esters such as methyl palmitate, fatty alcohols and mixtures thereof. Essential oils as core materials can include, for example, by way of illustration wintergreen oil, cinnamon oil, clove oil, lemon oil, lime oil, orange oil, peppermint oil and the like. Dyes can include fluorans, lactones, indolyl red, 16B, leuco dyes, all by way of illustration and not limitation. Other core materials include materials which alter rheology or flow characteristics of a product or extend shelf life or product stability.

As evident from the foregoing, the core materials include lipophilic/hydrophobic liquids as well as solid materials. Typically, though not necessarily, especially depending upon the core material itself, the core material is diluted with a diluent oil from 0.01 to 99.9 weight percent based on the combined weight of the diluent and core material in which it is dispersible or sufficiently soluble or miscible. In this regard, depending upon the specific core material and its use or purpose, the core material may be effective even at trace quantities, e.g., essential oils and fragrances. In following, the core material or benefit agent can be the majority or minority constituent encapsulated by the microcapsules.

In addition to the core material or benefiting agent, the oil phase, hence the microcapsule core, may also contain a partitioning modifier to aid encapsulation and retention of the core material or benefiting agent. The partitioning modifier can be the same material as the oil phase or diluent or can be different. The partitioning modifier can be selected from a larger group and can be further selected from the group consisting of oil soluble materials that have a C log P greater than from about 4, or from about 5, or from about 7, or even from about 11 and/or materials that also have a density higher than 1 gram per cubic centimeter.

### Microcapsule Formation

The microcapsules of the present teaching are formed by conventional methods for microcapsule formation, particularly conventional interfacial polymerization methods, with the exception that the isocyanates and gelatins are employed in the critical combinations, quantities and ratios as set forth above. Generally speaking, the oil phase comprising the isocyanate component and the core material is prepared by mixing, dissolving, etc., as appropriate, the core material in the isocyanate component, preferably at room temperature, until homogeneous. Concurrently, the water or aqueous phase is prepared by mixing, dissolving, etc., as appropriate, the gelatin in water, preferably deionized water, until fully dissolved or homogeneous. Depending upon the reactants and the desired pH, one may adjust the pH to that desired, e.g., by adding an acid such as acetic acid, hydrochloric acid, etc. Similarly, it may be desirable to warn the mixtures, perhaps up to 40° C. to aid in formation of the aqueous and oil phases, particularly where solubility of the ingredients (gelatin and/or core material, respectively) is poor. Once the two phases are complete, the oil phase is dispersed in the water phase under high shear agitation to form an oil-in-water emulsion comprising droplets of the oil phase monomer dispersed in the water phase. Typically, a high shear mixer blade is used and the size of the microcapsules is controlled by adjusting the speed and timing of agitation during the emulsion formation. Smaller size dispersions are the result of faster agitation. Once the desired droplet size is attained, the milling blade is then replaced with a propeller, stir bar, or like mixer blade and the reaction mix mixed while elevating the temperature to the desired reaction temperature and holding at that temperature for the necessary time period to ensure complete or near complete microcapsule formation. Of course, and preferably, the ramp up in temperature is a step-wise process where the temperature is ramped up to a first temperature over a stated period of time and held at that temperature before being ramped up to a second temperature, again over a given period of time, and held at that temperature until the reaction is completed or, as appropriate, performing one or more additional temperature ramp up and polymerization steps. Once the reaction is completed the reaction mix is allowed to return to room temperature after which the microcapsules are recovered by any of the well-known and well-practiced methods in the art.

Although heat alone is typically sufficient to complete the microencapsulation process and wall formation, one can speed up wall formation by the addition of suitable polymerization initiators and/or accelerators to the water phase. Suitable initiators include AIBN, sodium persulfate and benzoyl peroxide. When using initiators, the reaction temperature is elevated to whatever reaction temperature is appropriate for the initiator used. Additionally, as well recognized and known to those skilled in the art, one may use additional catalyst and/or pH adjustments to facilitate wall formation.

The thickness of the microcapsule wall is, in part, dependent upon the amount of the wall forming materials used. Generally, the wall forming material is from 0.1% to 40%, preferably from 0.1% to 20% based on the weight of the core composition to be encapsulated. Typical microcapsules formed in accordance with the present teaching will have a particle size of 0.1 to 150 microns, preferably from 0.5 to 100 microns, more preferably from 1 to 100 microns. Of course, different applications require larger or smaller particle sizes, even sized outside of the foregoing ranges.

As mentioned above, a number of other agents and additives may be present in the oil phase or water phase, particularly the latter, to aid in microcapsule formation and use. Exemplary additives include emulsifiers, deposition aids, initiators, pH adjusters, and the like.

Although the gelatin itself is found to aid in emulsification, it is often desirable to employ other emulsifiers. Such optional emulsifiers can be anionic, cationic, non-ionic and amphoteric emulsifiers. Generally preferred emulsifiers are the cationic and non-ionic emulsifiers, particularly those having poly (alkyl ether) units, especially polyethylene oxide units, with degrees of polymerization of the alkylene ether unit of greater than about 6. Preferred emulsifiers are those which significantly reduce the interfacial tension between the aqueous phase and oil phase, and thereby reduce the tendency for droplet coalescence. In this regard, generally the emulsifiers for use in the water phase for aiding in the oil in water emulsion or dispersion will have HLB values of from 8 to 20. Emulsifiers/surfactants of lower and higher HLB values that achieve the same objective may be employed.

For many emulsifiers, hydrophobic-lipophilic balance numbers (HLB) are reported in the literature and can be a useful guide in selection of the optional additional emulsifier. Exemplary emulsifiers and their HLB values are presented in Table 1.

TABLE 1

| Emulsifier | HLB value |
|---|---|
| Glycerol monostearate | 3.8 |
| Diglycerol monostearate | 5.5 |
| Tetraglycerol monostearate | 9.1 |
| Succinic acid ester of monoglycerides | 5.3 |
| Diacetyl tartaric acid ester of monoglycerides | 9.2 |
| Sodium stearoyl-2-lactylate | 21.0 |
| Sorbitan tristearate | 2.1 |
| Sorbitan monostearate | 4.7 |
| Sorbitan monooleate | 4.3 |
| Polyoxyethylene sorbitan monostearate | 14.9 |
| Propylene glycol monostearate | 3.4 |
| Polyoxyethylene sorbitan monooleate | 15.0 |

As noted, typical oil in water emulsifiers generally have an HLB (hydrophilic-lipophilic balance) value of 8 to 20, preferably 8 to 16. HLB values below about 8 generally are used to promote the water in oil emulsions. Optional emulsifiers of all types are suitable for use in the practice of the present teaching, though it is to be appreciated, and those skilled in the art will readily recognize, that different systems, i.e., different oil phase compositions, will be better suited with one or more classes of emulsifiers than others.

Additionally, a deposition aid may be added to the water phase, before, during or after formation of the microcapsule. The deposition aid is typically present in an amount of 0.1-10%, preferably 0.1-7.5% more preferably 0.1-5% wt %, based on the microcapsule solution. Deposition aids typically coat the outer surface of the shell of the microcapsule and aid in their use and application. Deposition aids can be coated onto capsules or covalently bonded, employing functional groups to effect linkage as generally described in Universidade do Minho (WO 2006117702); Gross et al. (U.S. Pat. Publ. No. 20170296440); and Devan Micropolis (U.S. Pat. Publ. No. 20080193761)

Exemplary deposition aids include poly(meth)acrylate, poly(ethylene-maleic anhydride), polyamine, wax, polyvinyl-pyrrolidone, polyvinylpyrrolidone co-polymers, polyvinylpyrrolidone-ethyl acrylate, polyvinylpyrrolidone-vinyl acrylate, polyvinyl-pyrrolidone methacrylate, polyvinyl-pyrrolidone-vinyl acetate, polyvinyl acetal, polyvinyl butyral, polysiloxane, poly(propylene maleic anhydride), maleic anhydride derivatives, co-polymers of maleic anhydride derivatives, polyvinyl alcohol, styrene-butadiene latex, gelatin, gum Arabic, carboxymethyl cellulose, carboxymethyl hydroxyethyl cellulose, hydroxyethyl cellulose, other modified celluloses, sodium alginate, chitosan, casein, pectin, modified starch, polyvinyl acetal, polyvinyl butyral, polyvinyl methyl ether/maleic anhydride, polyvinyl pyrrolidone and its co polymers, poly (vinylpyrrolidone-/methacrylamidopropyl trimethyl ammonium chloride), polyvinyl-pyrrolidone/vinyl acetate, polyvinyl pyrrolidone/dimethyl-aminoethyl methacrylate, polyvinyl amines, polyvinyl formamides, polyallyl amines and copolymers of polyvinyl amines, polyvinyl formamides, and polyallyl amines and mixtures thereof. Additional deposition aids include poly (acrylamide-co-diallyldimethylammonium chloride, poly (diallyldimethylammonium chloride, polyethylenimine, cationic polyamine, poly[(3-methyl-1-vinylimidazolium chloride)-co-(1-vinylpyrrolidone)], copolymer of acrylic acid and diallyldimethylammonium chloride, cationic guar, guar gum, an organopolysiloxane such as described in Gizaw et. al. (U.S. Pat. Publ. No. 20150030557), incorporated herein by reference.

Further, to aid in wall formation, one may add a variety of chemicals (borax, ammonium persulfate, epoxy resins, phosphate salts, etc.) to enhance cross-linking and to improve the physical and performance properties of the shell walls.

The microcapsules of the present teaching have, among other benefits, improved degradability due to the incorporation of the natural and bio-degradable gelatin polymer into the polyurethane/urea capsule wall. Additionally, as noted above, these microcapsules and their method of production enjoy a number of environmental, health and safety and economic benefits as a result of the marked reduction in isocyanate content and need. The microcapsules can be used dry or as a slurry of microcapsules, in coatings, as an additive to other materials, incorporated in or on fibers or textiles, or incorporated in or on polymeric materials, foams or other substrates. Optionally after microcapsule formation, the formed microcapsule can be isolated from the water phase or continuous phase, such as by decanting, dewatering, centrifuging, spray-drying, evaporation, freeze drying or other solvent removal or drying process.

The microcapsules of the invention can be incorporated dry, as an aqueous slurry, as a coating or as a gel into a variety of commercial products to yield novel and improved articles of manufacture, including incorporation into or onto foams, mattresses, bedding, cushions, added to cosmetics or to medical devices, incorporated into or onto packaging, dry wall, construction materials, heat sinks for electronics, cooling fluids, incorporated into insulation, used with lotions, incorporated into gels including gels for coating fabrics, automotive interiors, and other structures or articles, including clothing, footwear, personal protective equipment and any other article where use of the improved capsules of the invention is deemed desirable. Exemplary articles of manufacture include, but are not limited to soaps, surface cleaners, laundry detergents, fabric softeners, shampoos, textiles, coded dyes or pigments, paper products including carbonless record materials, tissues, towels, napkins, and the like, adhesives, wipes, diapers, feminine hygiene products, facial tissues, pharmaceuticals, deodorants, heat sinks, foams, pillows, mattresses, bedding, cushions, cosmetics and personal care products, medical devices, packaging, architectural coatings, surface treatments, pest repellents, paints, marine coatings, agricultural products including herbicides, fertilizers, and pesticides, coolants, wallboard, insulation, and the like. Blends of capsule populations can be useful, such as with differing sets of benefit agent, or even different wall formulations.

The microcapsules protect and separate the core material such as phase change material, fragrance, agricultural active, or other core material or benefit agent, keeping it separated from the external environment. This facilitates design of distinct and improved articles of manufacture. The microcapsules facilitate improving flowability of encapsulated materials enhancing ease of incorporation into or onto articles such as foams, gels, textiles, various cleaners, detergents or fabric softeners. For example, with phase change benefit agents, the microcapsules help preserve the repeated activity of the phase change material and retain the phase change material to prevent leakage or infusion into nearby components when isolation of the microcapsules is desired, yet promote eventual degradation of such encapsulates or portions of the articles of manufacture.

Having described the general and specific aspects of the present teaching, attention is now directed to the following examples.

### EXAMPLES

In the following examples, the abbreviations correspond to the following materials:

| Name | Company/City | Chemical Description |
|---|---|---|
| CAPTEX | Abitec, Columbus, | Caprylic/capric |
| 355 | OH | triglyceride (diluent) |
| DESMODUR | Covesto AG | hexamethylene |
| N3200A | | diisocyanate (HDI) |
| DESMODUR | Covesto AG | dicyclohexylmethane |
| W | | diisocyanate |
| FB | n/a | Fragrance blend |
| Gelatin | Sigma-Aldrich, Inc. | Fish gelatin |
| MONDUR | Covesto AG | diphenylmethane- |
| MR | | diisocyanate (MDI) |
| MONDUR | Covesto AG | polymeric MDI |
| MR LIGHT | | (methylene diphenyl |
| | | diisocyanate) |
| PVA 540 | Sekisui Specialty | Polyvinyl alcohol |
| | Chemicals America, LLC | |
| TAKENATE | Mitsui Chemicals Inc. | Xylylene diisocyanate |
| D-110N | | trimethylolpropane |
| | | adduct |

The core oil used in the examples is an equal part blend of Captex 355 (triglycerides of caprylic/capric acid) and fragrance blend. The fragrance blend (“FB”) employed in the examples is an equal part blend of benzyl acetate, octanal, linalool, 2,6-dimethyl-7-octen-2-ol, isobornyl acetate, linalyl acetate, butylphenyl methylpropional, isoamyl salicylate, and hexyl salicylate. All reference to parts in relation to the materials used in the preparation of the microcapsules is parts by weight.

### Test Methods

Several test methodologies were performed on the microcapsules. These test methods were for determining the particle size, free benefit agent, and leakage in hexane.

### Median Volume Weighted Particle Size

The volume-weighted median particle size of the microcapsules is measured using an Accusizer 780A, made by Particle Sizing Systems, Santa Barbara Calif., or equivalent. The instrument is calibrated from 0 to 300 μm (micrometer or micron) using particle size standards (as available from Duke/Thermo-Fisher-Scientific Inc., Waltham, Mass., USA). Samples for particle size evaluation are prepared by diluting about 0.5 g of microcapsule slurry in about 10 g of de-ionized water. This dilution is further diluted using about 1 g of the initially diluted solution in about 20 g of water. Approximately 1 g of the most dilute sample is injected into the Accusizer and the testing initiated using the autodilution feature. The Accusizer should read more than 8,500 counts/second. If the counts are below 8,500 additional sample is added. The sample is autodiluted until below 9,200 counts/second was measured, then particle counting, and size analysis is initiated. After 2 minutes of testing, the Accusizer displays the median volume-weighted particle size. Particle sizes stated herein are on a volume weighted basis and are to be understood as median volume weighted particle size, ascertainable by the above procedure.

### Percent Free Oil After Microencapsulation

Characterization of free oil in microcapsule suspension: 0.40-0.45 g of the microcapsule suspension is massed and mixed with 10 ml of hexane. The sample is mixed by vortexing at 3000 rpm for 10 seconds to leach the free oil from the microcapsule suspension and set aside for no more than one minute. An aliquot is removed from the hexane layer and filtered through a 0.45 μm syringe filter. The concentration of oil in the hexane is measured using an Agilent 7800 Gas Chromatograph (GC), Column: ZB-1HT (10 meter×0.32 mm×0.25 μm), Temp: 50° C. for 1 minute then heat to 270° C. @ 10° C./min, Injector: 275° C., Detector: 325° C., 2 μl injection.

### Degradability

Biodegradability of the microcapsules was determined in accordance with the OECD 301B test method. This test method classifies microcapsules as “Readily degradable” if the microcapsules show a degree of degradation of >60% in up to 28 days and as “Enhanced/modified readily biodegradable” if the degree of degradation is >60% in up to 60 days.

Leakage of Core Active into Liquid Matrices

The microcapsules were evaluated for release properties, i.e., release of the fragrance, in a heavy-duty liquid laundry detergent (HDL) from Seventh Generation, Inc. and in a commercial liquid fabric enhancer (LFE-unscented Downy® fabric softener).

The percent activity of the microcapsule slurry is calculated as the grams of benefit agent divided by grams of microcapsule slurry. The mass of the slurry needed for testing is then calculated as 1.5 divided by the percent activity. 50 g of the liquid matrix (HDL or LFE) is added to a glass jar. The appropriate mass of slurry is massed and placed in the jar containing liquid fabric enhancer under stirring until homogenized. The jar is capped and placed in an oven at 35° C. for one week. After one week the amount of free oil is measured. 0.4-0.5 g of the microcapsule suspension is massed, mixed with 2 mL RO water in a scintillation vial, and vortexed at 1,000 rpm for 60 second. 10 ml of hexane is added and vortexed at 1,000 rpm for 60 seconds. The sample is allowed to rest 30 minutes. An aliquot is removed from the hexane layer and filtered through a 0.45 μm syringe filter. The concentration of oil in the hexane is measured using an Agilent 7800 Gas Chromatograph (GC), Column: ZB-1HT (10 meter×0.32 mm×0.25 μm), Temp: 50° C. for 1 minute then heat to 270° C. @ 10° C./min, Injector: 275° C., Detector: 325° C., 2 μl injection.

### Determination of Release Properties

Characterization of the release properties of the core oil were measured using the CIPAC MT190 test method. The percent core agent after a one-hour extraction was normalized to the total concentration of the core agent contained in the microcapsule slurry.

### Example 1—Aromatic/Aliphatic Mid-Range Gelatin Microcapsules

A series of exemplary microcapsules (EMC) and comparative microcapsules (CMC) were prepared in accordance with the present teaching to demonstrate the impact of the use of a combination of aromatic isocyanates and combinations or aliphatic and aromatic isocyanates at different levels. The selection of wall forming components, core materials, and component weight ratios for each EMC and CMC were as presented in Table 2. The microcapsules were prepared by forming a water phase solution by dissolving 10 parts of fish gelatin from Sigma-Aldrich Inc. USA in 160 parts of deionized water in a reactor at ambient temperature resulting in a solution whose pH was 5. The capsule core composition was prepared in a beaker at ambient temperature by mixing the specified amount of the FB with the specified amount of Captex® 355 caprylic/capric triglyceride from ABITEC Corporation, which acted as a diluent for the FB. The specified amount of the one or more isocyanates was then added to the beaker under agitation by a mixer blade to complete the preparation of the oil phase. The oil phase was then gradually added to the reactor containing the water phase under high shear milling and the high shear milling maintained until the targeted emulsion droplet size of around 10 microns was attained.

TABLE 2

| | | | Isocyanate | | | | |
|---|---|---|---|---|---|---|---|
| | Core | | Takenate | Mondur | Desmodur | Desmodur | % Aliphatic |
| Microcap | FB | Captex | D110N | MR | W | N3200A | isocyanate |
| CMC-1 | 102 | 18 | 5.0 | 0.0 | | | |
| MC-2 | 102 | 18 | 3.75 | 1.25 | | | |
| MC-3 | 102 | 18 | 2.5 | 2.5 | | | |
| MC-4 | 102 | 18 | 0.0 | 5.0 | | | |
| MC-5 | 60 | 60 | 0.0 | 5.0 | | | |
| MC-6 | 60 | 60 | 5.0 | 0.0 | | | |
| MC-7 | 102 | 18 | 5.0 | 0.0 | | | |
| MC-8 | 102 | 18 | 2.5 | 0.0 | | | |
| EMC-1 | 102 | 18 | 5.0 | | 0.0 | | 0.0 |
| MC-9 | 102 | 18 | 3.75 | | 1.25 | | 25 |
| MC-10 | 102 | 18 | 2.5 | | 2.5 | | 50 |
| MC-11 | 102 | 18 | 0.0 | | 5.0 | | 100 |
| EMC-1 | 102 | 18 | 5.0 | | | 0.0 | 0.0 |
| MC-12 | 102 | 18 | 3.75 | | | 1.25 | 25 |
| MC-13 | 102 | 18 | 2.5 | | | 2.5 | 50 |
| MC-14 | 102 | 18 | 0.0 | | | 5.0 | 100 |

Once the target emulsion droplet size was achieved, milling was paused, and the milling blade switched out for a mixer blade to keep the emulsion mixed. Thereafter, the temperature of the reactor vessel was raised from ambient to 50° C. over a period of 60 min, and the temperature maintained at 50° C. for an additional 60 min. Subsequently, the temperature was then raised to 65° C. over 60 min, and maintained at 65° C. for an additional 120 min. Thereafter, the temperature was raised to 85° C. over a period of 60 min, and maintained at 85° C. for an additional four hours, at which time the process was deemed complete. The resulting microcapsule slurry in the reactor was allowed to cool to ambient temperature. The slurry was found to be of low viscosity and generally uniform and smooth with no visible thickening and/or agglomeration. The slurry and resulting microcapsules were then evaluated for free core material and leakage and biodegradation, respectively. These results are presented in Table 3.

TABLE 3

| | Leakage | | % | 301B % |
|---|---|---|---|---|
| Micro- | 7^{th} Generation | | Free | degradation |
| capsule | HDL | LFE | Core | 28 days |
| CMC-1 | 6.53 | 4.92 | 0.1 | 59.9 |
| MC-2 | 4.232 | 3.3 | 0.1 | |
| MC-3 | 6.69 | 7.82 | 0.1 | |
| MC-4 | 88.16 | 33.86 | 0.2 | |
| MC-5 | 34.61 | 28.97 | 0.1 | 63.33 |
| MC-6 | 9.86 | 7.02 | 0.11 | |
| MC-7 | 3.86 | 3.4 | 0.09 | 54.02 |
| MC-8 | 25.09 | 14.00 | 0.12 | |
| EMC-1 | 6.53 | 4.92 | 0.1 | 59.9 |
| MC-9 | 4.6 | 4.4 | 0.1 | |
| MC-10 | 17.15 | 9.34 | 0.1 | |
| MC-11 | 92.93 | 48.24 | 0.5 | |
| EMC-1 | 6.53 | 4.92 | 0.1 | 59.9 |
| MC-12 | 5.18 | 4.8 | 0.1 | |
| MC-13 | 14.57 | 15.32 | 0.1 | |
| MC-14 | 67.59 | 46.52 | 0.4 | |

### Example 2—Aliphatic/Aromatic Diisocyanate/Gelatin Microcapsules

A second series of microcapsules (MC) according to the present teaching were formed using different combinations of aliphatic and aromatic diisocyanates at different levels relative to the amount of fish gelatin. The selection of wall forming components, core materials, and component weight ratios microcapsule were as presented in Table 3. The microcapsules were prepared by forming a water phase solution by adding the specified amount of fish gelatin from Sigma-Aldrich Inc. USA to the specified amount of deionized water with mixing using a 4-tip mill at 1000 rpm in a temperature controlled/jacketed reactor at 20° C. temperature until the gelatin is fully dissolved. The pH of the water phase was adjusted to 3.5 using hydrochloric acid: 2.5 in the case of MC-25. Separately, the capsule core composition was prepared by mixing 85 parts of the specified core agent with 85 parts Captex® 355 caprylic/capric triglyceride from ABITEC Corporation, which acted as a diluent for the core agent: the only exception being microcapsule MC-23 where 51 parts FB was mixed with 34 parts of the diluent. Thereafter, the specified amount of the isocyanates was added to the core composition with mixing until a homogeneous mixture was attained. Thereafter, the oil phase is gradually added to the aqueous phase under milling at 500 rpm and, once fully added, the milling speed increased to 1200 rpm and maintained at that rate until the targeted droplet size is attained, typically about 20 microns, preferably 16 microns or less: generally, ˜10-30 minutes. The milling blade was replaced with a three-inch propeller blade and the mixture stirred at 300 rpm. The pH of the reaction mix was then adjusted to 6.0 using sodium hydroxide: pH 5.0 in the case of microcapsules MC-25 and MC-26. The temperature of the reaction mix was then increased to 50° C. over a period of 60 minutes and maintained at 50° C. for an additional 60 minutes. Thereafter, the temperature was increased to 65° C. over a period of 60 minutes and maintained at 65° C. for an additional 6 hours, except in the case of microcapsules MC-22 and MC-25 where the final cure temperature was maintained for 4 rather than 6 and microcapsules MC-22 MC-26 where the final cure step was conducted at 85° C. Following expiration of the cure period, heating was terminated and the reaction mix allowed to return to room temperature naturally.

### Example 3—Microcapsule Series

A series of microcapsules, MC-A through MC-L, were prepared in accordance with general procedure of Example 2 but varying the amounts of the wall forming materials and, to a lesser extent, the reaction conditions. With the exception of MC-H and MC-K, for which the final cure period was 4 hours, the timing of Example 2 was followed. Similarly, with the exception of MC-H and MC-L for which the final cure temperature was 85° C., the cure temperatures of Example 2 were followed. The make-up of the aqueous and oil phases is presented in Table 4: the core in all but MC-E through MC-G and MC-I was a 50:50 mix of Captex 355 and FB. MC-E through MC-G were 50:50 mixtures of Captex 355 and a fragrance, a dye and an essential oil, respectively, and MC-I was a 60:40 mix of FB: Captex 355. The properties of the resulting microcapsules are presented in Table 5. As noted, there is some variation on milling time to achieve the desired or targeted droplet size. For example, in MC-A, a mill time of 7 minutes resulted in droplets of Vol-Wt median size of 24.7 microns and continuing milling for a total of 14 minutes resulted in droplets of 20.78 microns. The final microcapsule size was 23.36 microns with 42.5% solids and 17.78% of the FB.

TABLE 4

| | | | | Mondur | | | % | |
|---|---|---|---|---|---|---|---|---|
| | | | Core | MR | Desmodur | Mondor | Aliphatic | Wt Ratio |
| Microcap | water | gelatin | active | Light | N3200A | W | isocyanate | gelatin:isocyanate |
| MC-15 | 293.66 | 28.34 | FB | 1.58 | 3.02 | | 58.3 | 1:0.16 |
| MC-16 | 293.66 | 28.34 | FB | 1.51 | 3.1 | | 60 | 1:0.16 |
| MC-17 | 293.66 | 28.34 | FB | 1.08 | 2.22 | | 60 | 1:0.11 |
| MC-16 | 293.66 | 28.34 | FB | 0.65 | 1.33 | | 60 | 1:0.07 |
| MC-19 | 293.66 | 28.34 | Fragrance | 1.58 | 3.02 | | 58.3 | 1:0.16 |
| MC-20 | 293.66 | 28.34 | Dye | 1.58 | 3.02 | | 58.3 | 1:0.16 |
| MC-21 | 293.66 | 28.34 | Essential | 1.58 | 3.02 | | 58.3 | 1:0.16 |
| | | | oil | | | | | |
| MC-22 | 297.85 | 24.15 | FB | 3.24 | 4.43 | | 50.0 | 1:0.32 |
| MC-23 | 277.56 | 44.44 | FB | 2.06 | 2.35 | | 45.55 | 1:0.1 |
| MC-24 | 293.66 | 28.34 | FB | 0.38 | 4.66 | | 90.0 | 1:0.18 |
| MC-25 | 297.85 | 24.15 | FB | 10.69 | 1.63 | | 10.0 | 1:0.5 |
| MC-26 | 281.75 | 40.25 | FB | 10.69 | | 1.18 | 10.0 | 1:0.29 |
| MC-27 | 293.66 | 28.34 | FB | 0.0 | 5.17 | | 100 | 1:0.18 |

All documents cited in the specification herein are, in relevant part, incorporated herein by reference for all jurisdictions in which such incorporation is permitted. The citation of any publication is for its disclosure prior to the filing date and should not be construed as an admission that such publication is prior art or that the present invention is not entitled to antedate such publication by virtue of prior invention. To the extent that any meaning or definition of a term in this document conflicts with any meaning or definition of the same term in a document incorporated by reference, the meaning or definition assigned to that term in this document shall govern. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

TABLE 5

| | | | | | | Ag | 301B |
|---|---|---|---|---|---|---|---|
| | | | | | | Release | Degradation |
| | Milling | Cure | LFE | HDL | Free Core | (% at 60 | (% after 14 |
| Microcap | pH | pH | Leakage (%) | Leakage (%) | (% of slurry) | minutes) | days) |
| MC-15 | 3.5 | 6.0 | 10.7 | N/A | 0.1 | 50.8 | N/A |
| MC-16 | 3.5 | 6.0 | 22.4 | N/A | 0.1 | 59.0 | N/A |
| MC-17 | 3.5 | 6.0 | 37.0 | N/A | 0.1 | 64.4 | N/A |
| MC-18 | 3.5 | 6.0 | 29.9 | N/A | 0.1 | 65.5 | N/A |
| MC-19 | 3.5 | 6.0 | 3.71 | 6.24 | 0.1 | N/A | 53.5 |
| MC-20 | 3.5 | 6.0 | N/A | N/A | 0.0 | N/A | 52.3 |
| MC-21 | 3.5 | 6.0 | 12.3 | 19.4 | 0.2 | N/A | 33.0 |
| MC-22 | 3.5 | 6.0 | 6.94 | N/A | 0.1 | 44.4 | N/A |
| MC-23 | 3.5 | 6.0 | 18.54 | N/A | 0.1 | 67.38 | N/A |
| MC-24 | 3.5 | 6.0 | 33.46 | N/A | 0.1 | 65.5 | N/A |
| MC-25 | 2.5 | 5.0 | 11.87 | N/A | 0.1 | 49.12 | N/A |
| MC-26 | 3.5 | 5.0 | 14.75 | N/A | 0.1. | 57.15 | 48.9 |
| MC-27 | 3.5 | 6.0 | 70.11 | N/A | 0.1 | 70.77 | N/A |

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. No unclaimed language should be deemed to limit the invention in scope. Any statements or suggestions herein that certain features constitute a component of the claimed invention are not intended to be limiting unless reflected in the appended claims.

The principles, preferred embodiments, and modes of operation of the present invention have been described in the foregoing specification. The invention which is intended to be protected herein, however, is not to be construed as limited to the particular forms disclosed, since these are to be regarded as illustrative rather than restrictive variations and charges can be made by those skilled in the art without departing from the spirit and scope of the invention.

We claim:

1. A microcapsule formed by a non-coacervate oil-in-water microencapsulation process, said microcapsule comprising a core material and a polymer shell encapsulating the core material wherein the polymer shell comprises the polymerization/-copolymerization reaction product of gelatin and an isocyanate component wherein the isocyanate component comprises a mixture of at least one aliphatic di- and/or poly-isocyanate or a biuret or adduct thereof and at least one aromatic di- and/or poly-isocyanates or a biuret or adduct thereof, said isocyanates being copolymerizable with the gelatin, wherein the weight ratio of gelatin to isocyanate is from 1:0.5 to 1:0.01 and the mole ratio of the aliphatic isocyanate to the aromatic isocyanate is from 65:30 to 10:90.

2. The microcapsule of claim 1 wherein the weight ratio of gelatin to isocyanate is from 1:0.35 to 1:0.05.

3. The microcapsule of claim 1 wherein the weight ratio of gelatin to isocyanate is from 1:0.20 to 1:0.1.

4. The microcapsule of claim 1 wherein the mole ratio of the aliphatic isocyanate to the aromatic isocyanate is from 60:40 to 20:80.

5. The microcapsule of claim 1 wherein the mole ratio of the aliphatic isocyanate to the aromatic isocyanate is from 60:40 to 30:70.

6. The microcapsule of claim 1 wherein the mole ratio of the aliphatic isocyanate to the aromatic isocyanate is from 65:30 to 50:50.

7. The microcapsule of claim 1 wherein at least 50 mole percent of the required isocyanates are di-isocyanates or a biuret or adduct thereof.

8. The microcapsule of claim 1 wherein one or more of the required isocyanates is a biuret and/or an adduct.

9. The microcapsule of claim 1 wherein the gelatin has a molecular weight of from about 5,000 to about 80,000.

10. The microcapsule of claim 1 wherein the non-coacervate oil-in-water microencapsulation process involves the steps of i) forming an oil phase composition comprising the core material and the isocyanate component and a water phase composition comprising a homogeneous solution of a wall-forming gelatin dissolved in water, ii) subsequently combining the oil phase with the water phase and subjecting the mixture to high shear agitation for a period of time to form an oil-in-water emulsion comprising droplets of the oil phase composition of a desired droplet size in the water phase, iii) thereafter subjecting the emulsion, while mixing, to an elevated temperature sufficient to induce shell wall formation through the polymerization/copolymerization of the isocyanate and gelatin components, with or without a polymerization initiator and/or accelerator and with or without a cross-linking agent, and iv) allowing the reaction to continue for a sufficient period of time whereby the desired microcapsules are formed.

11. A non-coacervate oil-in-water microencapsulation process for preparing a microcapsule comprising a core material and a polymer shell encapsulating the core material wherein the polymer shell comprises the polymerization/copolymerization reaction product of gelatin and an isocyanate component wherein the isocyanate component comprises a mixture of at least one aliphatic di- and/or poly-isocyanate or a biuret or adduct thereof, and at least one aromatic di- and/or poly-isocyanates or a biuret or adduct thereof, said isocyanates being copolymerizable with the gelatin, wherein the weight ratio of gelatin to isocyanate is from 1:0.5 to 1:0.01 and the mole ratio of the aliphatic isocyanate to the aromatic isocyanate is from 65:30 to 10:90, said process comprising i) forming an oil phase composition comprising the core material and the isocyanate component and a water phase composition comprising water and a wall-forming gelatin dissolved therein, ii) subsequently combining the oil phase with the water phase and subjecting the mixture to high shear agitation for a period of time to form an oil-in-water emulsion comprising droplets of the oil phase composition of a desired droplet size in the water phase, iii) thereafter subjecting the emulsion to an elevated temperature sufficient to induce shell wall formation through the polymerization/copolymerization of the isocyanate component and the gelatin, with or without a polymerization initiator and/or accelerator and with or without a cross-linking agent, and iv) allowing the reaction to continue for a sufficient period of time whereby the desired microcapsules are formed.

12. The process of claim 11 wherein the gelatin content in the water phase is from about 0.1 to about 40% by weight based on the combined weight of the gelatin and the water and the isocyanate component comprises from 0.1 to 10 percent by weight of the oil phase.

13. The process of claim 11 wherein the gelatin content in the water phase is from about 1 to about 10% by weight based on the combined weight of the gelatin and the water and the isocyanate component comprises from 1.5 to 3.5 percent by weight of the oil phase.

14. The process of claim 11 wherein the weight ratio of gelatin to isocyanate is from 1:0.35 to 1:0.05.

15. The process of claim 11 wherein the weight ratio of gelatin to isocyanate is from 1:0.20 to 1:0.1.

16. The process of claim 11 wherein the mole ratio of the aliphatic isocyanate to the aromatic isocyanate is from 60:40 to 20:80.

17. The process of claim 11 wherein the mole ratio of the aliphatic isocyanate to the aromatic isocyanate is from 60:40 to 30:70.

18. The process of claim 11 wherein at least 50 mole percent of the required isocyanates are di-isocyanates or a biuret or adduct thereof.

19. The process of claim 11 wherein one or more of the required isocyanates is a biuret and/or an adduct thereof.

20. The process of claim 11 wherein the gelatin has a molecular weight of from about 5,000 to about 80,000.
